(19) United States
(12) Patent Application Publication
(10) Pub. No.: US 2004/0241818 A1
(43) Pub. Date: Dec. 2, 2004

(54) Assay

(76) Inventors: David John Hayes, Dundee (GB); Martin John Sims, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,594

(22) PCT Filed: Oct. 17, 2001
(86) PCT No.: PCT/EP01/11949

(30) Foreign Application Priority Data
Oct. 20, 2000 (GB) 0025804.6

(51) Int. Cl. C12N 9/12

(52) U.S. Cl. 435/194

(57) ABSTRACT

This invention relates to truncated Tec kinase polypeptides and their use in screening for compounds which modulate the activity of Tec kinase polypeptides. Also described are nucleotide sequences encoding truncated Tec kinase polypeptides, vectors and host cells containing said nucleotides.

### FIELD OF INVENTION

The present invention relates to truncated Tec kinase polypeptides, nucleotide sequences encoding truncated Tec kinase polypeptides, vectors and host cells containing said nucleotides, methods of screening for compounds which modulate the activity of Tec kinase polypeptides, and the use of compounds identifiable by said method in therapy.

### BACKGROUND TO THE INVENTION

Antigen receptors on T, B and mast cells are multimolecular complexes that are activated by interactions with external signals. These signals are then transmitted to regulate gene expression and posttranscriptional modifications. A family of non-receptor tyrosine kinases known as the “Tec kinase family”, including Itk, Tec, Btk, Bmx and Txk tyrosine kinases, is involved in the signal transduction in T, B and mast cells. The members of the Tec kinase family share a similar domain structure, having an N-terminal pleckstrin-homology (PH) domain; a Tec homology domain (TH), which includes one (Itk, Bmx, Txk, and Tec29) or two (Btk and Tec) proline-rich regions (PR); Src homology 3 (SH3) and Src homology 2 (SH2) domains; and a catalytic kinase domain (SH1), see for exampie Yang et al. (2000) Immunity 12:373-382.

A need exists to identify modulators of Tec kinase polypeptides in order to provide compounds for use in therapy. At present, there are no methods available for screening Tec kinase polypeptides. Accordingly, the object of the present invention is to provide a screen for compounds which modulate the activity of Tec kinase polypeptides and thereby provide compounds for use in therapy.

### SUMMARY OF INVENTION

The present invention is based on the finding that a truncated Tec kinase polypeptide can be used in an assay to screen for compounds which modulate the activity of Tec kinase polypeptides.

Accordingly, the invention provides a truncated Tec kinase polypeptide having a Tec kinase amino acid sequence truncated by a minimum of the amino acids constituting the PH domain and a portion of the TH domain including at least one proline rich region up to but not including the amino acids constituting the kinase domain.

Another aspect of the invention is an isolated polynucleotide which (a) encodes a truncated Tec kinase polypeptide of the invention; (b) is complementary to polynucleotide (a); (c) selectively hybridises to polynucleotide (a) or (b); or (d) is degenerate as a result of the genetic code from polynucleotide (a), (b) or (c).

Further aspects of the invention are:

an expression vector which comprises a polynucleotide of the invention and which is capable of expressing a polypeptide of the invention;

a host cell comprising an expression vector of the invention;

a primer which is capable of generating a polynucleotide of the invention;

a method of producing a polypeptide of the invention which method comprises introducing into an appropriate cell line a vector comprising a polynucleotide of the invention under conditions suitable for obtaining expression of the polypeptide;

a method for the identification of a compound which modulates the activity of a Tec kinase polypeptide, comprising contacting a polypeptide of the invention with a test compound and detecting any enhancement or inhibition in the activity of the polypeptide, compared to the activity that would occur in the absence of said test compound;

a compound which modulates Tec kinase activity and is identifiable by the method referred to above;

use of a compound which modulates Tec kinase activity and is identifiable by the method referred to above for the manufacture of a medicament for the treatment or prophylaxis of a disorder that is responsive to modulation of Tec kinase activity; and

a method of treating a subject having a disorder that is responsive to modulation of Tec kinase activity, which method comprises administering to said subject an effective amount of a compound identifiable by the method referred to above.

### DETAILED DESCRIPTION OF THE INVENTION

We have surprisingly found that a truncated Tec kinase polypeptide, which is truncated by a minimum of the amino acids constituting the PH domain and at least one of the proline rich region of the TH domain, up to but not including the amino acids constituting the kinase domain, is suitable for screening for compounds which modulate the activity of Tec kinase polypeptides. The truncated Tec kinase polypeptides of the present invention are constitutively active despite truncation of a large portion of the protein.

The polypeptides of the invention have been found to be particularly suitable for screening as they do not need to be pre-activated by phosphorylation. In vivo, Tec kinases need to be phosphorylated by other kinases in order to activate the enzyme (Gibson et al. (1996) J. Immunology 156:2716-2722). Whilst pre-activation by phosphorylation is commonly required in assays, for example, LCK assays include a phosphorylation step (Trevillyan, et al (1999) Arch. Biochem. Biophys. 364, 19-29), the removal of the need to preactivate the polypeptides of the present invention offers a simplification for the assay.

A further advantage of the present invention is the provision of an assay that is “robust”. The present inventors have found that the assay is robust in two respects. Firstly, it is possible to generate large amounts of truncated enzyme which are stable over a long time. Secondly, the assay gives a high frequency of comparable results upon repeat testing.

The polypeptides of the invention may therefore provide useful screening targets for the identification and development of novel pharmaceutical agents, including agonists and antagonists of Tec kinases, which may be useful in the treatment and/or prophylaxis of disorders such as inflammation.

Truncated Tec Kinase Polypeptides

The invention provides a truncated Tec kinase polypeptide having a Tec kinase amino acid sequence truncated by a minimum of the amino acids constituting the PH domain and a portion of the TH domain including at least one proline rich region up to but not including the amino acids constituting the kinase domain. In other words, the point of truncation can occur anywhere after the first proline rich region of the TH domain (i.e. the proline rich region closest to the PH domain) and before the kinase domain. Preferably, the Tec kinase amino acid sequence is truncated by a minimum of the amino acids constituting the PH domain and a portion of the TH domain including at least one proline rich region up to but not including the amino acids constituting the SH2 and kinase domain, i.e. the point of truncation can occur anywhere after the first proline rich region of the TH domain and before the SH2 domain. More preferably, the Tec kinase amino acid sequence is truncated by the amino acids constituting PH domain and a portion of the TH domain including at least one proline rich region up to but not including the amino acids constituting the SH3, SH2 and kinase domain, i.e. the point of truncation can occur anywhere after the first proline rich region of the TH domain and before the SH3 domain. In a preferred aspect of the invention, when the Tec kinase polypeptide contains more than one proline rich region, the truncated Tec kinase polypeptide does not contain any proline rich regions, i.e. the point of truncation occurs after all the proline rich regions of the TH domain.

The domains of the Tec kinase polypeptides referred to above are defined in FIGS. 5 and 6. FIG. 6 shows a schematic representation of the domain structure of Tec kinases.

The “PH domain” as described herein is the N-terminal domain of the Tec kinase polypeptide. It comprises β sheet structure.

The “TH domain” as described herein is the domain situated between the PH and SH3 domains of the Tec kinase polypeptide. It comprises a globular core and either one (Itk, Bmx, Txk, Tec29) or two (Tec, Btk) proline rich (PR) regions.

The “proline rich regions” as described herein are the proline rich regions in the TH domain. Preferably, a proline rich region comprises at least two proline residues within six consecutive amino acids, preferably at least three proline residues within six consecutive amino acids, more preferably at least four proline residues within six consecutive amino acids.

The “SH3 domain” as described herein is the domain situated between the TH and SH2 domains of the Tec kinase polypeptide. It comprises two P sheets positioned at approximately right angles to each other.

The “SH2 domain” as described herein is the domain situated between the SH3 and kinase domain of the Tec kinase polypeptide.

The “kinase domain” as described herein is the C-terminal domain of the Tec kinase polypeptide. The “kinase domain” may alternatively be known as the “SH1” domain.

A “Tec kinase polypeptide” as described herein is a polypeptide of the Tec kinase family, including Itk, Tec, Btk, Bmx and Txk tyrosine kinases.

FIG. 5 shows an alignment of the polypeptide sequences of Tec family kinases showing the PH, TH, SH3 and SH2 domains. The ClustaIX analysis program was used to align the sequences (Thompson, J. D., Gibson, T. J., Plewniak, F., Jeanmougin, F. and Higgins, D. G. (1997) The ClustalX windows interface: flexible strategies for multiple sequence alignment aided by quality analysis tools. Nucleic Acids Research, 24:4876-4882). The default settings were used.

Accordingly, in a preferred aspect of the invention, the Tec kinase polypeptide is truncated at a position between amino acid 199 (last amino acid of the first proline rich region of the TH domain, when the sequence is aligned as shown in FIG. 5) and amino acid 451 (first amino acid of the kinase domain, when the sequence is aligned as shown in FIG. 5). More preferably the Tec kinase polypeptide is truncated at a position between amino acid 199 (last amino acid of the first proline rich region of the TH domain, when the sequence is aligned as shown in FIG. 5) and amino acid 417 (last amino acid of the SH2 domain, when the sequence is aligned as shown in FIG. 5). More preferably the Tec kinase polypeptide is truncated at a position between amino acid 199 (last amino acid of the first proline rich region of the TH domain, when the sequence is aligned as shown in FIG. 5) and amino acid 333 (first amino acid of the SH2 domain, when the sequence is aligned as shown in FIG. 5). Even more preferably the Tec kinase polypeptide is truncated at a position between amino acid 199 (last amino acid of the first proline rich region of the TH domain, when the sequence is aligned as shown in FIG. 5) and amino acid 224 (first amino acid of the SH3 domain, when the sequence is aligned as shown in FIG. 5).

In a more preferred aspect of the invention, when the Tec kinase polypeptide has more than one proline rich region, the Tec kinase polypeptide is truncated at a position between amino acid 213 (last amino acid of the second proline rich region of the TH domain, when the sequence is aligned as shown in FIG. 5) and amino acid 451 (first amino acid of the kinase domain, when the sequence is aligned as shown in FIG. 5). More preferably the Tec kinase polypeptide is truncated at a position between amino acid 213 (last amino acid of the second proline rich region of the TH domain, when the sequence is aligned as shown in FIG. 5) and amino acid 417 (last amino acid of the SH2 domain, when the sequence is aligned as shown in FIG. 5). More preferably the Tec kinase polypeptide is truncated at a position between amino acid 213 (last amino acid of the second proline rich region of the TH domain, when the sequence is aligned as shown in FIG. 5) and amino acid 333 (first amino acid of the SH2 domain, when the sequence is aligned as shown in FIG. 5). Even more preferably the Tec kinase polypeptide is truncated at a position between amino acid 213 (last amino acid of the second proline rich region of the TH domain, when the sequence is aligned as shown in FIG. 5) and amino acid 224 (first amino acid of the SH3 domain, when the sequence is aligned as shown in FIG. 5).

In a particular aspect of the invention, the start of the SH3 domain is defined by the amino acid sequence: *X-X-V-[VIK]-A-[LM]-Y-D-[YF]; the start of the SH2 domain is defined by the amino acid sequence: [IL]-[ED]-X-Y-E-*W-Y; and the start of the kinase domain is defined by the amino acid sequence: G-[LF]-[GRS]-Y-[GDE]-[SK]-W-*, where,

the letters denote amino acids in one letter code,

the square brackets denote a single amino acid,

the amino acids within the square brackets are alternatives,

X is any one amino acid residue, and

“*” indicates the start of the domain.

For example, the Tec kinase polpeptide sequences to be truncated may be selected from Btk (human)—Accession no. Q06187 (SwissProt), Btk (mouse)—Accession no. P35991 (SwissProt), Itk (human)—Accession no. Q08881 (SwissProt), Itk (mouse)—Accession no. Q03526 (SwissProt), Tec (human)—Accession no. P42680 (SwissProt), Tec (mouse)—Accession no. P24604 (SwissProt), Bmx (human)—Accession no. P51813 (SwissProt), Bmx (mouse)—Accession no. P97504 (TREMBL), Txk (human)—Accession no. P42681 (SwissProt), and Txk (mouse)—Accession no. P42682 (SwissProt).

Preferably the truncated Tec kinase polypeptide is a truncated Itk polypeptide or a truncated Btk polypeptide. Preferably the Itk polypeptide to be truncated has a sequence as set forth in FIG. 4 (“ITK” sequence) or a homolog or variant thereof. Preferably the truncated ltk polypeptide has the polypeptide sequence set forth in FIG. 3 or a homolog or variant thereof. More preferably, the truncated Itk polypeptide has a sequence encoded by the polynucleotide sequence set forth in FIG. 2. Preferably the Btk polypeptide to be truncated has a sequence Btk (human) Accession no. 006187 (Swiss prot. Database) or a homolog or variant thereof. Preferably, the truncated Btk polypeptide has the polypeptide sequence set forth in FIG. 8 or a homolog or variant thereof. More preferably, the truncated Btk polypeptide has a sequence encoded by the polynucleotide sequence set forth in FIG. 9.

The polypeptides of the present invention are provided in an isolated form. The term “isolated” is intended to convey that the material is not in its native state. Thus, the naturally-occurring polypeptide present in a living animal is in its native state and is not isolated, but the same polypeptide, separated from some or all of the materials it co-exists with in the natural system, is isolated. The polypeptides may be mixed with carriers or diluents which will not interfere with their intended use and still be regarded as isolated. Similarly, a polypeptide which has been produced by synthetic means, for example, by recombinant methods is “isolated”. The polypeptides of the present invention are also preferably provided in purified form and preferably are purified to at least 50% purity, more preferably about 75% purity, most preferably 90% purity or greater, such as 95%, 98% pure. Routine methods can be employed to purify and/or synthesize the polypeptides according to the invention. Such methods are well understood by persons skilled in the art, and include techniques such as those disclosed in Sambrook et al, Molecular Cloning: a Laboratory Manual, 2^{nd} Edition, CSH Laboratory Press, 1989, the disclosure of which is included herein in its entirety by way of reference.

The polypeptide of the present invention may be a recombinant or a synthetic polypeptide. The polypeptide of the invention is a human or animal sequence (or homologous to such sequence). Such an animal is typically a mammal, such as a rodent (e.g. a mouse) or a primate. Preferably the polypeptide is a human sequence.

Homologues of polypeptide sequences are referred to above. Such homologues typically have at least 70% homology, preferably at least 80%, 90%, 95%, 97% or 99% homology, for example over a region of at least 15, 20, 30, 100 or more contiguous amino acids. The homology may be calculated on the basis of amino acid identity (sometime referred to as “hard homology”). Identity is calculated using the widely used GCG (University of Wisconsin) suite of programs and preferably using the distances software (correction method).

The term “variant” refers to a polypeptide which has a same essential character or basic biological functionality as the truncated Tec kinase polypeptide in question. Preferably a variant polypeptide is one which binds to the same ligand as the truncated Tec kinase polypeptide. Such variants may include allelic variants and the deletion, modification or addition of single amino acids or groups of amino acids within the polypeptide sequence.

Amino acid substitutions may be made, for example from 1, 2 or 3 to 10, 15, 20 or 30 substitutions. The modified polypeptide retains activity as a truncated Tec kinase polypeptide. Changes in amino acid sequence of peptides can be guided by known similarities among amino acids and other molecules or substituents in physical features such as charge density, hydrophobicity, hydrophilicity, size and configuration etc. For example the amino acid Thr may be replaced by Ser and vice versa, and Leu may be replaced by lie and vice versa. For example, a polar amino acid such as glycine or serine may be substituted for another polar amino acid; a basic amino acid may be substituted for another basic amino acid; an acidic amino acid may be substituted for another acidic amino acid; or a non-polar amino acid may be substituted for another non-polar amino acid. Groups of amino acids normally considered to be equivalent are:

| | |
|---|---|
| (a) | Ala (A), Ser (S), Thr (T), Pro (P), Gly (G); |
| (b) | Asn (N), Asp (D), Glu (E), Gln (Q); |
| (c) | His (H), Arg (R) Glu (E), Gln (Q); |
| (d) | Met (M), Leu (L), Ile (I), Val (V); and |
| (e) | Phe (F), Tyr (Y), Trp (W). |

Polypeptides of the invention may be chemically modified, e.g. post-translationally modified. For example, they may be glycosylated or comprise modified amino acid residues. They may also be modified by the addition of histidine residues to assist their purification, or other suitable protein tags, see for example, Nilsson et al. (1997) Protein Expression and Purification 11:1-16. Such modified polypeptides fall within the scope of the term “polypeptide” of the invention.

PolynLicleotides and Primers

A further aspect of the invention is an isolated polynucleotide which (a) encodes a truncated Tec kinase polypeptide of the invention; (b) is complementary to polynucleotide (a); (c) selectively hybridises to polynucleotide (a) or (b); or (d) is degenerate as a result of the genetic code from polynucleotide (a), (b) or (c). A preferred aspect is an isolated polynucelotide which: (a) encodes the truncated ltk polypeptide set forth in FIG. 3; (b) is complementary to polynucleotide (a); (c) selectively hybridises to polynucleotide (a) or (b); or (d) is degenerate as a result of the genetic code from polynucleotide (a), (b) or (c). A more preferred aspect of the invention is an isolated polynucleotide having (a) the sequence set forth in FIG. 2; (b) a sequence complementary to polynucleotide (a); (c) a sequence which selectively hybridises to polynucleotide (a) or (b); or (d) a sequence that is degenerate as a result of the genetic code from polynucleotide (a), (b) or (c). A further aspect of the invention, is an isolated polynucelotide which: (a) encodes the truncated Btk polypeptide set forth in FIG. 8; (b) is complementary to polynucleotide (a); (c) selectively hybridises to polynucleotide (a) or (b); or (d) is degenerate as a result of the genetic code from polynucleotide (a), (b) or (c). A more preferred aspect of the invention is an isolated polynucleotide having (a) the sequence set forth in FIG. 9; (b) a sequence complementary to polynucleotide (a); (c) a sequence which selectively hybridises to polynucleotide (a) or (b); or (d) a sequence that is degenerate as a result of the genetic code from polynucleotide (a), (b) or (c).

The polynucleotide sequences of the present invention may be in the form of RNA or in the form of DNA, for example cDNA, genomic DNA, and synthetic DNA. Preferably the polynucleotide sequence of the invention is cDNA. The DNA may be double-stranded or single-stranded, and if single stranded may be the coding strand or non-coding (anti-sense) strand.

As used herein the term polynucleotide includes nucleic acids that contain one or more modified (e.g., tritylated) or unusual (e.g., inosine) bases. Thus, DNAs or RNAs with backbones modified for stability or for other reasons are polynucleotides as that term is intended herein.

The polynucleotide of the invention is a human or animal sequence (or homologous to such sequence). Such an animal is typically a mammal, such as a rodent (e.g. a mouse) or a primate. Preferably the polynucleotide is a human sequence.

Homologues of polynucleotide sequences typically have at least 70% sequence identity, preferably at least 80%, 90%, 95%, 97% or 99% sequence identity, for example over a region of at least 15, 20, 30, 100 or more contiguous nucleotides. Methods of measuring nucleic acid homology are well known in the art. For example, the UWGCG Package provides the BESTFIT program which can be used to calculate homology (Devereux et al 1984). Similarly the PILEUP and BLAST algorithms can be used to line up sequences (for example are described in Altschul 1993, and Altschul et al 1990). In accordance with the invention, the default settings may be used.

As used herein, “selective hybridisation” means that generally the polynucleotide can hybridize to the gene region sequence at a level significantly above background. The signal level generated by the interaction between a polynucleotide of the invention and the gene region sequence is typically at least 10 fold, preferably at least 100 fold, as intense as interactions between other polynucleotides and the gene region sequence. The intensity of interaction may be measured, for example, by radiolabelling the polynucleotide, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C).

The coding sequence of polynucleotides of the present invention may be modified by nucleotide substitutions, for example from 1, 2 or 3 to 10, 15, 25, 50 or 100 substitutions. The polynucleotide may alternatively or additionally be modified by one or more insertions and/or deletions and/or by an extension at either or both ends. The modified polynucleotide encodes a polypeptide which has the activity associated with a truncated Tec kinase polypeptide. Degenerate substitutions may be made and/or substitutions may be made which would result in a conservative amino acid substitution when the modified sequence is translated, for example as shown above.

Generation of a Tec kinase oligonucleotide may be performed by methodologies known in the art such as polymerase chain reaction (PCR) for example on genomic DNA or cDNA with appropriate oligonucleotide primers derived from or designed based on a knowledge of the sequence of the Tec kinase of interest. For example, the polynucleotide sequences of Tec kinases may be selected from: Btk (human)—Accession no. X58957 (EMBL/GenBank), Btk (mouse)—Accession no. L08967 (EMBL/GenBank), Itk (human)—Accession no. D13720 (EMBL/GenBank), Itk (mouse)—Accession no. L00619 (EMBL/GenBank), Tec (human)—Accession no. D29767 (EMBL/GenBank), Tec (mouse)—Accession no. S53716 (EMBL/GenBank), Bmx (human)—Accession no. X83107 (EMBL/GenBank), Bmx (mouse)—Accession no. U88091 (EMBL/GenBank), Txk (human)—Accession no. L27071 (EMBL/GenBank), and Txk (mouse)—Accession no. U16145 (EMBL/GenBank).

According to a further aspect of the invention, there is provided a primer which is capable of generating a Tec kinase polynucleotide which encodes a truncated Tec kinase polypeptide of the invention. An allele specific primer is used, generally together with a constant primer, in an amplification reaction such as a PCR reaction, which provides discrimination between alleles through selective amplification of one allele at a particular sequence position. The allele specific primer is preferably at least 10, preferably at least 15 or at least 20, for example at least 25, at least 30 nucleotides in length. For example, in the generation of a truncated ltk polypeptide, the primers shown in FIG. 1 may be used to generate the corresponding polynucleotide. In the generation of a truncated Btk polypeptide, the primers shown in FIG. 7 may be used to generate the corresponding polynucleotide.

The polynucleotide sequences of the present invention are provided in an isolated form. The term “isolated” is intended to convey that the material is not in its native state. Thus, the naturally-occurring polynucleotide sequence present in a living animal is in its native state and is not isolated, but the same polynucleotide sequence, separated from some or all of the materials it co-exists with in the natural system, is isolated. They may be mixed with carriers or diluents which will not interfere with their intended use and still be regarded as isolated. Such polynucleotide sequence could be part of a vector. Such polynucleotide'sequence could be part of a composition, and still be isolated in that such vector or composition is not part of its natural environment. The polynucleotide sequences of the present invention are also preferably provided in purified form, and preferably are purified to at least 50% purity, more preferably about 75% purity, most preferably 90% purity or greater, such as 95%, 98% pure.

Production of Tec Kinase Polypeptides

The polynucleotide sequences of the present invention may be employed for producing a polypeptide of the invention by recombinant techniques. Thus, for example the nucleotide sequence may be included in any one of a variety of expression vehicles or cloning vehicles, in particular vectors or plasmids for expressing a protein.

A further aspect of the invention is therefore a vector comprising a polynucleotide of the invention. Appropriate cloning and expression vectors for use with prokaryotic and eukaryotic hosts include mammalian expression vectors, insect expression vectors, yeast expression vectors, bacterial expression vectors and viral expression vectors, see Sambrook et al., Molecular Cloning: A Laboratory Manual, Second Edition, Cold Spring Harbor, N.Y., (1989). Examples of suitable vectors include derivatives of bacterial plasmids; phage DNA; yeast plasmids; vectors derived from combinations of plasmids and phage DNA and viral DNA and baculoviruses. A preferred vector is a baculovirus.

In a preferred aspect, the vector further comprises one or more regulatory sequences to direct mRNA synthesis, including, for example, a promoter, operably linked to the sequence. Suitable promoters include: insect cell promoters, polyhedrin promotor, p10 and other promoters known to control expression of genes in prokaryotic or eukaryotic cells or their viruses. The vector may contain an enhancer and a ribosome binding site for translation initiation and a transcription terminator. Large numbers of suitable vectors and promoters/enhancers, will be known to those of skill in the art, but any plasmid or vector, promoter/enhancer may be used as long as it is replicable and functional in the host. The vector may also include appropriate sequences for selection and/or amplification of expression. For this the vector will comprise one or more phenotypic selectable/amplifiable markers. Such markers are also well known to those skilled in the art.

In a further aspect, the present invention provides host cells comprising a vector of the invention, and capable of expressing a nucleotide sequence of the invention. The host cells can be, for example, a higher eukaryotic cell, such as a mammalian cell or a lower eukaryotic cell, such as a yeast cell or a prokaryotic cell such as a bacterial cell. Suitable prokaryotic hosts for transformation include E-coli. Suitable eukaryotic hosts include insect cells, e.g. SF9 cells. Tni cells, Hi5 cells, and mammalian cells, e.g. HEK HeLa, COS, CHO, NSO 3T3 (fibroblast cell line). Cells expressing a nucleotide sequence of the invention can be lysed to obtain the polypeptide of the invention, which may optionally be purified.

Cell free translation systems can also be employed to produce such proteins using RNAs derived from the DNA constructs of the present invention.

A further aspect of the invention is a method of producing a polypeptide of the invention, which method comprises introducing into an appropriate cell line a vector comprising a polynucleotide as defined herein under conditions suitable for obtaining expression of the polypeptide.

Methods of Screening

The present invention further provides a method for identification of a compound which modulates Tec kinase activity. Compounds which modulate Tec kinase activity are those which inhibit or enhance the function of the Tec kinase polypeptides (e.g. to act as antagonists or agonists of the Tec kinase protein function). In general terms, the screen for such compounds will comprise contacting a polypeptide of the invention with a test compound, and then detecting any enhancement or inhibition of polypeptide activity that results (compared to the activity that would occur in the absence of the test compound). By contacting, it is meant that the test compound and the polypeptide of the invention are in such proximity that they are able to interact biologically. The polypeptides of the invention may be used in high throughput screens, thus enabling large numbers of compounds to be studied.

The activity that is detected is preferably phospho-transfer activity. Phospho-transfer involves the transfer of a phosphate from ATP to a tyrosine residue contained within the substrate. Phospho-transfer activity may be detected by the inclusion of a target polypeptide in the assay. More particularly, polypeptides of the invention may be used in any suitable tyrosine kinase assay, for example, by a time-resolved fluorescence assay, such as homogenous time-resolved fluorescence (HTRF , Packard Instrument Company) Kolb et al., DDT Vol. 3, 333-342; in-plate binding assays including calorimetric and luminescent read-outs and time-resolved fluorescence, see for example Farley et al., Anal Biochem (1992) 203, 151-157, Lehel et al., (1997) Anal. Biochem. 244, 340-346 and Braunwalder et al., (1996) Anal. Biochem. 238, 340-346; or radiometric assays, for example, using ³³P or ³²P, such as scintillation proximity assay (SPA, Amersham International). Examples of time-resolved fluorescence assays are: in-plate time-resolved fluorescence (trf) assay (Delfia®, Wallac Oy). Preferably a time-resolved fluorescence assay is used, most preferably homogenous time-resolved fluorescence.

In a preferred aspect, the method of screening is carried out using cells expressing a polypeptide of the invention, and incubating such cells with the test compound, optionally in the presence of a Tec kinase ligand.

Methods of Treatment

A further aspect of the present invention is a compound which modulates Tec kinase activity and is identifiable by screening techniques referred to above. Preferably, the compound has been identified using the above screening techniques. The compounds may be agonists or antagonists of the Tec kinase polypeptide, but preferably are antagonists. The compounds include, for example, aptamers, polypeptides, antibodies and small molecules. The compounds may be useful in the treatment and/or prophylaxis of disorders that are responsive to modulation of Tec kinase activity. For example, it has been demonstrated in vivo that Itk-deficient mice are unable to establish TH2 cells suggesting that Itk has a role in mediating the development of IL-4 producing TH2 cells (Fowell et al. (1999) Immunity 11:399-409). The absence of this cytokine in Itk-deficient mice suggests that compounds which modulate Tec kinases may be useful in the treatment of inflammatory diseases such as, for example, asthma.

One particular aspect of the invention is the use of a compound which modulates Tec kinase activity and is identifiable by the screening techniques of the invention, for the manufacture of a medicament for the treatment or prophylaxis of disorders that are responsive to modulation of the activity of Tec kinase activity, such as inflammation.

Another aspect of the invention is a method of treating a subject having a disorder which is responsive to modulation of Tec kinase activity, such as inflammation, which method comprises administering to a subject an effective amount of a compound identifiable by the screening techniques of the invention.

Examples of inflammatory conditions include: asthma, allergic rhinitis, URID (upper respiratory inflammatory disease), adult respiratory distress syndrome; arthritic conditions such as rheumatoid arthritis, rheumatoid spondylitis, and osteoarthritis; inflammatory eye conditions such as uveitis (including iritis) and conjunctivitis; inflammatory bowel conditions such as Crohn's disease, ulcerative colitis and distal proctitis; periodontal disease; esophagitis, inflammatory skin conditions such as psoriasis, eczema and dermatitis. Preferred inflammatory conditions are asthma, allergic rhinitis and URID (upper respiratory inflammatory disease).

Where the Tec kinase is Btk, preferably the disorder is selected from inflammatory conditions (described above); diseases with a B cell component such as B cell leukaemias and SLE (Systemic Lupus erythematosis); and diseases associated with platelet function.

Substances identified according to the screening methods outlined above may be formulated with standard pharmaceutically acceptable carriers and/or excipients as is routine in the pharmaceutical art. The exact nature of a formulation will depend upon several factors including the particular substance to be administered and the desired route of administration. The substances may be administered by enteral or parenteral routes such as via oral, buccal, anal, pulmonary, intravenous, intra-arterial, intramuscular, intraperitoneal, topical or other appropriate administration routes.

Therapeutically effective amounts of such compounds can be readily determined by those skilled in the art using, e.g. dose-response studies. The dose of agent may be determined according to various parameters, especially according to the substance used; the age, weight and condition of the patient to be treated; the route of administration and the required regime. A suitable dose may however be from 0.01 to 50 mg/kg body weight such as 1 to 40 mg/kg body weight. A physician will be able to determine the required route of administration and dosage for any particular patient.

### BRIEF DESCRIPTION OF THE FIGURES:

FIG. 1 shows the oligonucleotide sequences used to generate the truncated Itk construct shown in FIG. 2.

FIG. 2 shows the polynucleotide sequence of the truncated Itk construct (including cloning sites, underlined).

FIG. 3 shows the polynucleotide and translated polypeptide sequence of the truncated ltk construct.

FIG. 4 shows an alignment of the polypeptide sequences of Itk (as in Swissprot data base accession number: Q08881) and the truncated construct showing PH; SH3; SH2 and kinase domains. The domain boundaries are as defined by Swissprot entry Q08881: PH 4-111; SH3 171-23; SH2 239-338; Kinase 363-615. Note: tITK=truncated ITK.

FIG. 5 shows an alignment of the polypeptide sequences of Tec family kinases (SwissProt database):

Btk (human)—Accession no. Q06187, Btk (mouse)—Accession no. P35991, Itk (human)—Accession no. 008881, Itk (mouse)—Accession no. Q03526, Tec (human)—Accession no. P42680, Tec (mouse)—Accession no. P24604, Bmx (human)—Accession no. P51813, Txk (human)—P42681/Q14220, and Txk (mouse)—Accession no. P42682, showing the PH domain (Bold, italic and boxed), Tec homology domain (Italic and boxed), SH3 domain (Bold and boxed) and SH2 domain (Boxed).

FIG. 6 shows a schematic representation of the domain structure of Tec kinases.

FIG. 7 shows the oligonucleotide sequences used to generate the truncated Btk construct shown in FIG. 9.

FIG. 8 shows the translated polypeptide sequence of the truncated Btk construct.

FIG. 9 shows the polynucleotide sequence of the truncated Btk construct. Note: the full length sequence is available on the genembl database accession 20 number X58957.

### EXAMPLES

### Example 1A

Generation of the Truncated Itk Construct

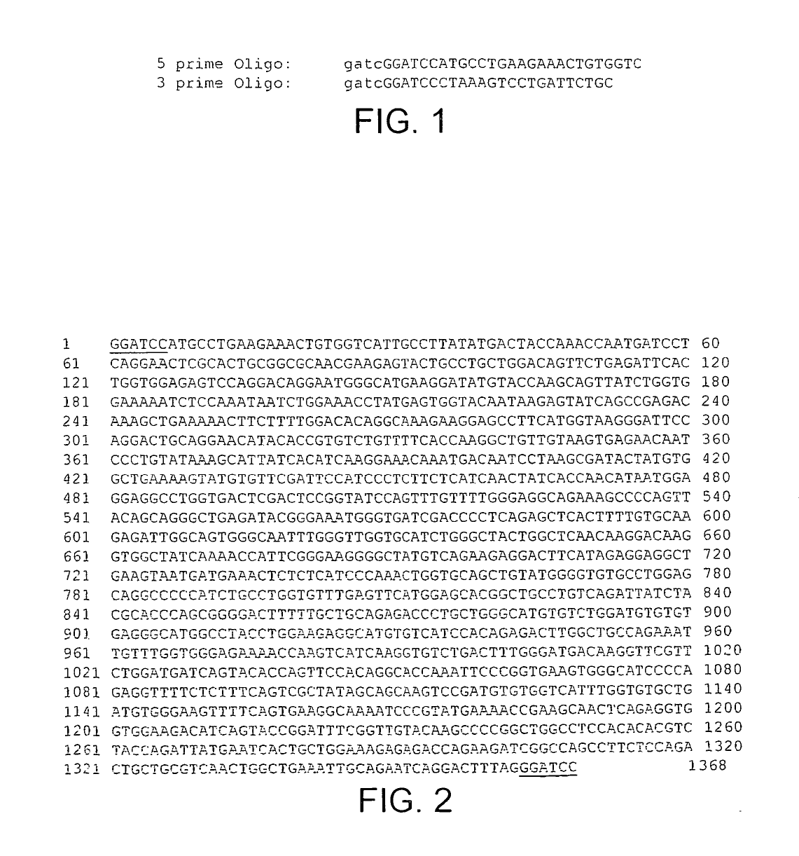

To generate an active form of Itk oligonucleotide, PCR primers were designed to amplify, from cDNA, a single region of Itk corresponding to the combined SH3, SH2 and kinase domains and to incorporate a start methionine and restriction endonuclease sites for cloning the construct. The Oligonucleotide sequences used to generate the truncated Itk construct are shown in FIG. 1. A T cell cDNA library was used as a source of template DNA (generation of library described in Biotechniques (1998) 25:85-92). A PCR product was cloned (FIG. 2). sequenced and used to generate a recombinant baculovirus for infection of SF9 insect cells using standard molecular biological techniques (see for example, Sambrook et al, Molecular Cloning: a Laboratory Manual, 2_{nd} Edition, CSH Laboratory Press, 1989).

### Example 1 B

Protein Expression and Assay Formation

Insect cell pellets infected with the recombinant baculovirus described in Example 1A were homogenised in 40 mM HEPES (pH 7.4), 100 mM NaCl 2 mM EDTA, 10% glycerol, 0.1 mM vanadate and protease inhibitors. The 100 000 g supernatant was stored at −85° C. Stored lysates were thawed on ice, ATP and MgCl₂ (0.1 mM and 10 mM) were added. Following incubation on ice the kinase was diluted in 40 mM HEPES (pH 7.4). The kinase reaction mixture contained 40 mM HEPES (pH 7.4). 10 mM MgCl₂, 0.05 mM ATP, 0.0005 mM peptide (Biotin-AAAEEIYGEI). The reaction was stopped by the addition of EDTA (25 mM). The amount of phosphopeptide was quantitated by homogeneous time resolved fluorescence as described in Kolb et al. (1998) Drug Discovery Today 3:333-342. An increase in the level of fluorescence indicates an increase in kinase activity.

The Km for ATP and peptide was determined to be 0.039+/−0.011mM and 0.480+/−0.183 uM respectively.

### Example 1 C

### Screening Compounds for Modulation of Itk Activity

The table below shows inhibitors of Itk activity identified using the screen described in Example 1B above.

Compound Testing in Kinase Assay

Order of additions:

Compound to well

Enzyme to well

15 min pre-incubation

Substrates (ATP, src-peptide) added

30 min incubation

Reaction stopped with EDTA

HTRF reagents (APC, Eu labelled antibody (antiPY antibody)) added

Stand for 20 mins

Read signal on plate reader

| | | UK:ITK | |
|---|---|---|---|
| | | Average | Average |
| | Class Name | PIC50 | IC50 (μM) |
| 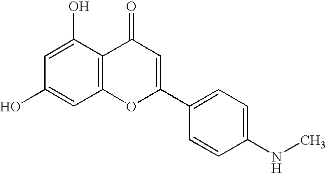 | flavone | 6.09 | 0.813 |
| 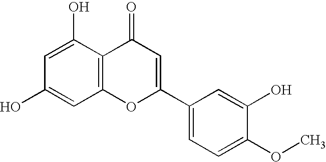 | flavone | 6.09 | 0.813 |
| 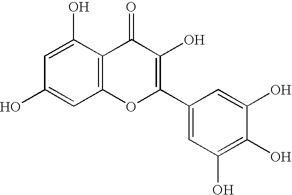 | flavone | 5.78 | 1.660 |

Note: PIC₅₀ is -log10 IC₅₀ in molar, a higher PIC₅₀ indicates greater potency.

### Example 1 D

Performance of Itk Screen

The following data demonstrate the robustness of the Itk assay. Repetition of the assay described above over a period between 12, Aug. 1999 and 24, Feb. 2000 (Batch 1) and 22, May 2000 and 25, May 2000 (Batch 2) demonstrates: (i) it is possible to generate large amounts of truncated enzyme which are stable over a long time, and (ii) the assay gives a high frequency of comparable results upon repeat testing.

| | Batch 1 | | Batch 2 | |
|---|---|---|---|---|
| Date | Aug. 12, | Feb. 24, 2000 | Mar. 22, 2000 | May 25, 2000 |
| | 1999 | | | |
| Control | 7076 ± 735 | 6373 ± 461 | 6978 ± 341 | 5616 ± 66 |
| Blank | 175 ± 7 | 193 ± 31 | 140 ± 15 | 120 ± 3 |
| Signal/ | 40 | 33 | 50 | 47 |
| Noise | | | | |

The results are expressed as the mean ± SD (n ≧ 80).

### Example 2A

Generation of the Truncated Btk Construct

To generate an active form of Btk oligonucleotide, PCR primers were designed to amplify, from cDNA, a single region of Btk corresponding to the combined SH3, SH2 and kinase domains and to incorporate a start methionine and restriction endonuclease sites for cloning the construct. The Oligonucleotide sequences used to generate the truncated Btk construct are shown in FIG. 7. A PCR product was cloned, sequenced and used to generate a recombinant baculovirus for infection of SF9 insect cells using standard molecular biological techniques (see for example, Sambrook et al, Molecular Cloning: a Laboratory Manual, 2^{nd} Edition, CSH Laboratory Press, 1989).

### Example 2B

Protein Expression and Assay Formation

Insect cell pellets infected with the recombinant baculovirus described in Example 2A were homogenised in 40 mM HEPES (pH 7.4), 100 mM NaCl 2 mM EDTA, 10% glycerol, 0.1 mM vanadate and protease inhibitors. The 100 000 g supernatant was stored at −85° C. Stored lysates were thawed on ice and diluted in 40 mM HEPES (pH 7.4). The kinase reaction mixture contained 40mM HEPES pH7.4; 80 mM MgCl₂; 1300 μM ATP; 500 nM peptide (Biotin-AAAEEIYGEI-NH2). The reaction was stopped by the addition of EDTA (0.16 M). The amount of phosphopeptide was quantitated by homogeneous time resolved fluorescence as described in Kolb et al. (1998) Drug Discovery Today 3:333-342. An increase in the level of fluorescence indicates an increase in kinase activity.

The Km for ATP was determined to be 1.69+/−0.36 mM

### Example 2C

Screening Compounds for Modulation of Btk Activity

The table below shows an inhibitor of Btk activity identified using the screen described in Example 2B above.

Compound Testing in Kinase Assay

Order of additions:

Compound to well

Enzyme to well

15 min pre-incubation

Substrates (ATP, src-peptide) added

30 min incubation

Reaction stopped with EDTA

HTRF reagents (APC. Eu labelled antibody (antiPY antibody)) added

Stand for 20 mins

Read signal on plate reader

Compound: Staurosporin

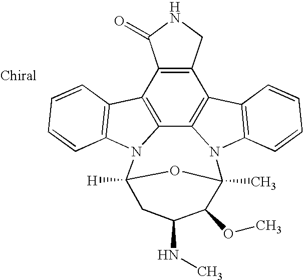

pIC₅₀=6.83

Note: PIC₅₀ is −log10 IC₅₀in molar, a higher PIC₅₀ indicates greater potency.

1. A method for the identification of a compound which modulates the activity of a Tec kinase polypeptide, comprising contacting (i) a truncated Tec kinase polypeptide wherein the Tec kinase amino acid sequence does not contain the amino acids constituting the PH domain and a portion of the TH domain including at least one proline rich region, but does contain the amino acids constituting the kinase domain with (ii) a test compound, and detecting any enhancement or inhibition in the activity of the polypeptide, compared to the activity that would occur in the absence of said test compound.

2. A method as claimed in claim 1 wherein the Tec kinase amino acid sequence contains the amino acids constituting the SH2 and kinase domain.

3. A method as claimed in claim 1 wherein the Tec kinase amino acid sequence contains the amino acids constituting the SH3, SH2 and kinase domain.

4. A method according to claim 1 wherein the truncated Tec kinase polypeptide does not contain any proline rich regions.

5. A method according to claim 1 wherein the Tec kinase polypeptide is Itk.

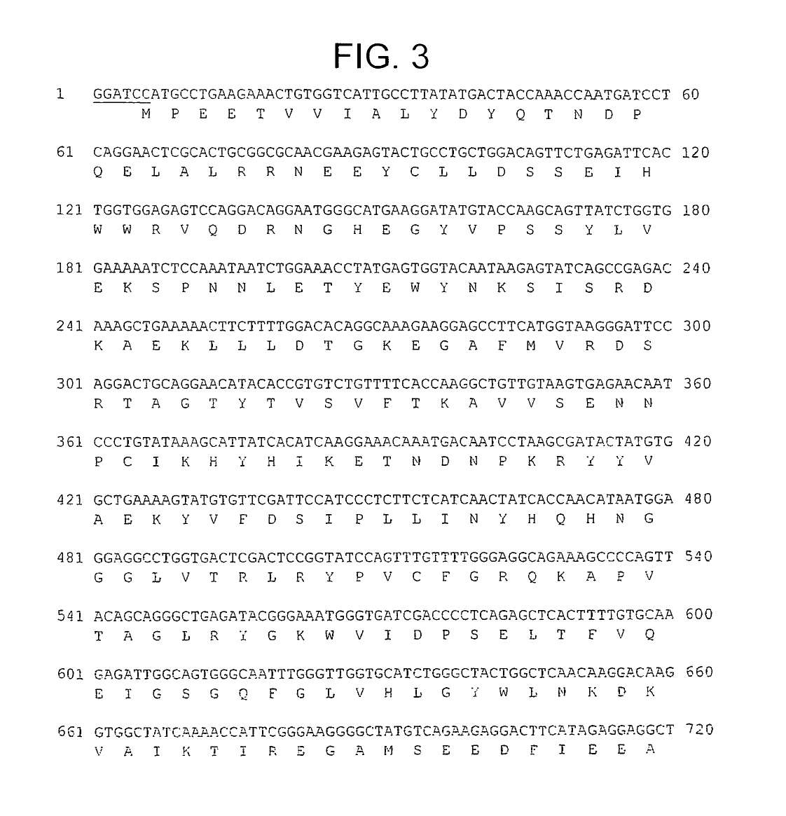

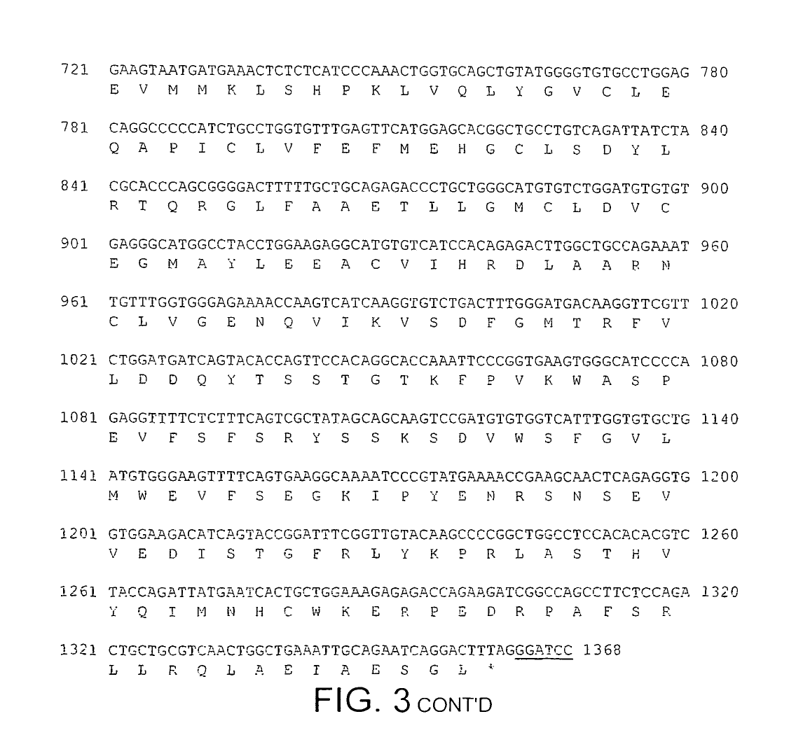

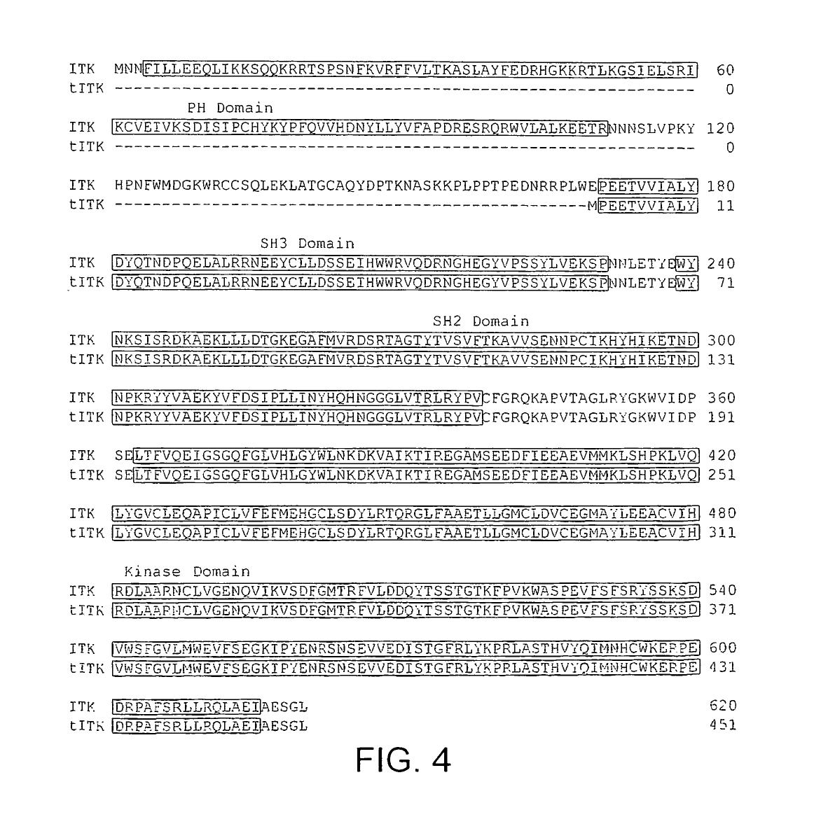

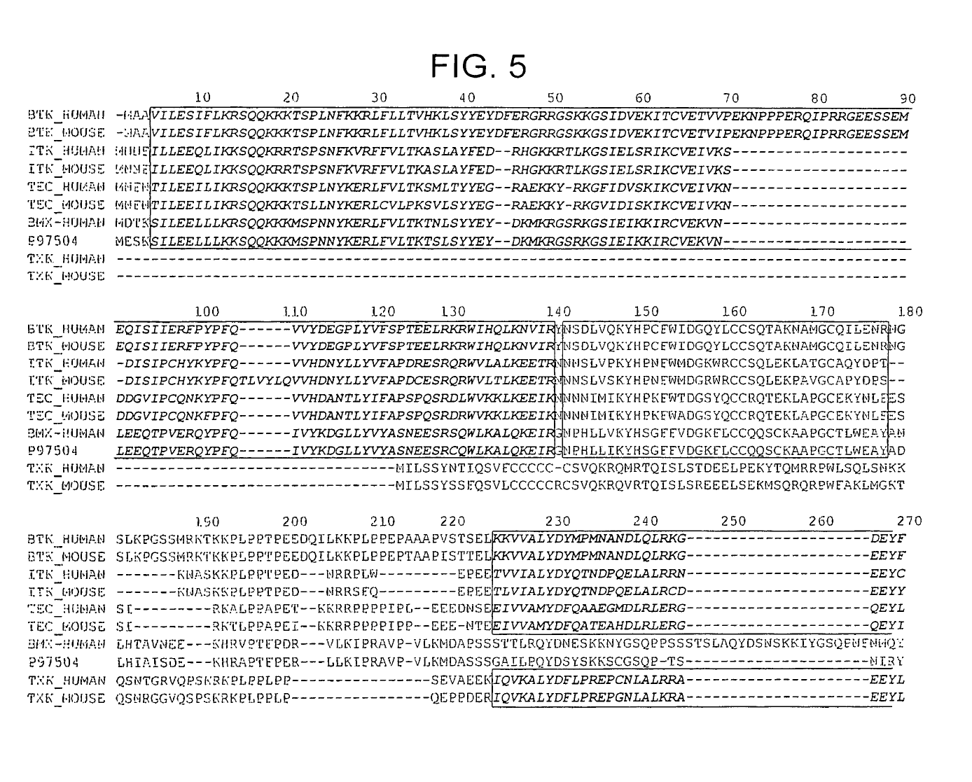

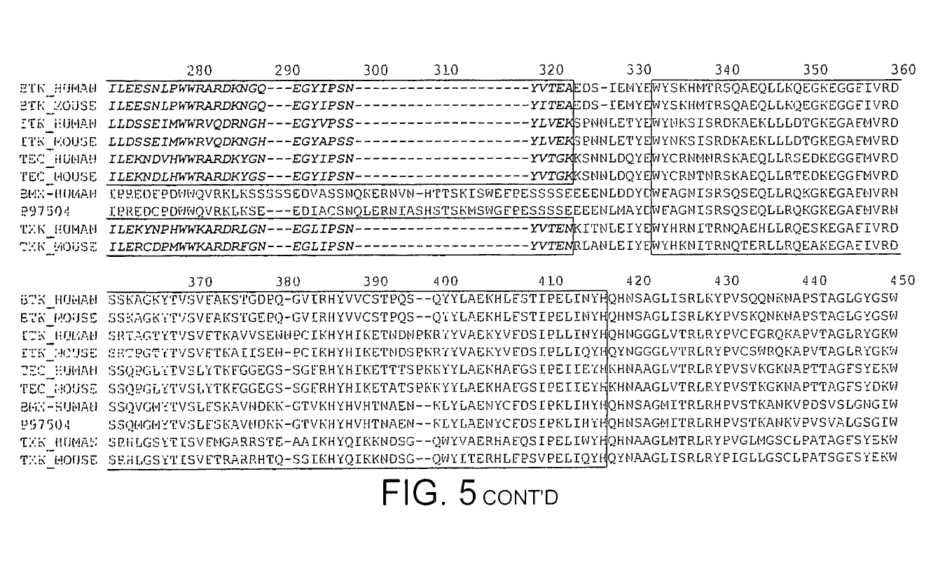

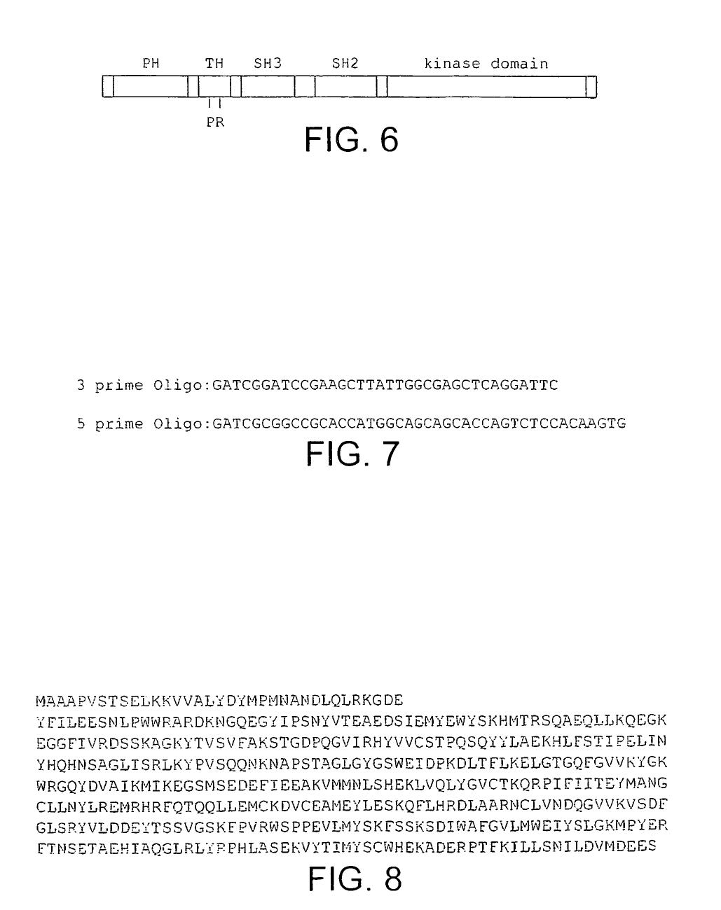

6. A method as claimed in claim 5 wherein the Tec kinase polypeptide has an amino acid sequence selected from the sequence of FIG. 3 and sequences having at least 70% homology thereto.

7. A method as claimed in claim 1 wherein the Tec kinase polypeptide is Btk.

8. A method as claimed in claim 7 wherein the Tec kinase polypeptide has an amino acid sequence selected from the sequence set forth in FIG. 8 and sequences having at least 70% homology thereto.

9. A method for the identification of a compound as claimed in claim 1, comprising contacting a compound of interest with the truncated Tec kinase polypeptide in the presence of a target polypeptide and observing any enhancement or inhibition of phosphorylation of the target polypeptide.

10. A method according to claim 1 wherein a cell line expressing the truncated Tec kinase polypeptide is contacted with the test compound.

11. A truncated Tec kinase polypeptide having a Itk kinase amino acid sequence that does not contain the amino acids constituting the PH domain and a portion of the TH domain including at least one proline rich region, but does contain the amino acids constituting the kinase domain.

12. A truncated Tec kinase polypeptide having a Tec kinase amino acid sequence that does not contain the amino acids constituting the PH domain and a portion of the TH domain including at least one proline rich region, but does contain the amino acids constituting the SH2 and kinase domain.

13. A truncated Tec kinase polypeptide as claimed in claim 12 wherein the Tec kinase amino acid sequence contains the amino acids constituting the SH3, SH2 and kinase domain.

14. A truncated Tec kinase polypeptide as claimed in claim 12 which does not contain any proline-rich regions.

15. A truncated Tec kinase polypeptide as claimed in claim 11, which does not contain any proline-rich regions.

16. A truncated Tec kinase polypeptide as claimed in claim 11 having an amino acid sequence selected from (a) the sequence set forth in FIG. 3 and (b) sequences having at least 70% homology thereto.

17. A turmcated Tec kinase polypeptide as claimed in claim 12 wherein the Tec kinase polypeptide is Btk.

18. A truncated Tec kinase polypeptide as claimed in claim 17 having an amino acid sequence selected from (a) the sequence set forth in FIG. 8 and (b) sequences having at least 70% homology thereto.

19. An isolated polynucleotide which:
(a) encodes a truncated Tec kinase polypeptide according to claim 1;
(b) is complementary to polynucleotide (a);
(c) selectively hybridises to polynucleotide (a) or (b); or
(d) is degenerate as a result of the genetic code from polynucleotide (a), (b) or (c).

20. An isolated polynucelotide as claimed in claim 19 which:
(a) encodes the truncated Itk polypeptide set forth in FIG. 3;
(b) is complementary to polynucleotide (a);
(c) selectively hybridises to polynucleotide (a) or (b); or
(d) is degenerate as a result of the genetic code from polynucleotide (a), (b) or (c).

21. An isolated polynucleotide as claimed in claim 20 having:
(a) the sequence set forth in FIG. 2;
(b) a sequence complementary to polynucleotide (a);
(c) a sequence which selectively hybridises to polynucleotide (a) or (b); or
(d) a sequence that is degenerate as a result of the genetic code from polynucleotide (a), (b) or (c).

22. An isolated polynucelotide as claimed in claim 19 which:
(a) encodes the truncated Btk polypeptide set forth in FIG. 8;
(b) is complementary to polynucleotide (a);
(c) selectively hybridises to polynucleotide (a) or (b); or
(d)is degenerate as a result of the genetic code from polynucleotide (a), (b) or (c).

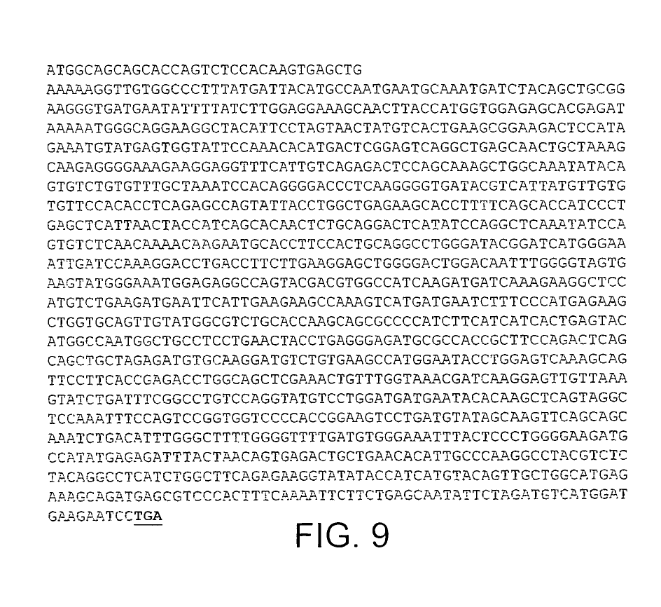

23. An isolated polynucleotide as claimed in claim 22 having:
(a) the sequence set forth in FIG. 9;
(b) a sequence complementary to polynucleotide (a);
(c) a sequence which selectively hybridises to polynucleotide (a) or (b); or
(d) a sequence that is degenerate as a result of the genetic code from polynucleotide (a), (b) or (c).

24. A vector comprising a polynucleotide according to claim 19.

25. A host cell comprising a vector as claimed in claim 24.

26. (Cancel)

27. A method of producing a polypeptide, which method comprises introducing into an appropriate cell line a vector comprising a polynucleotide as claimed in claim 9 under conditions suitable for obtaining expression of the polypeptide encoded by said polynucleotide.

28. (Cancel)

29. (Cancel)

30. (Cancel)

31. (Cancel)


SEQUENCE LISTING (23 sequences)

SEQ ID NO: 1 - protein, 9 aa, Artificial Sequence
  SITE (1)..(2): Xaa is any one amino acid residue
XXVXAXYDX

SEQ ID NO: 2 - protein, 7 aa, Artificial Sequence
  SITE (1): Xaa is Ile or Leu
XXXYEWY

SEQ ID NO: 3 - protein, 7 aa, Artificial Sequence
  SITE (2): Xaa is Leu or Phe
GXXYXXW

SEQ ID NO: 4 - DNA, 31 bp, Artificial Sequence
  Description of Artificial Sequence 5 prime oligo
gatcggatccatgcctgaagaaactgtggtc

SEQ ID NO: 5 - DNA, 28 bp, Artificial Sequence
  Description of Artificial Sequence 3 prime oligo
gatcggatccctaaagtcctgattctgc

SEQ ID NO: 6 - DNA, 1368 bp, Artificial Sequence
  CDS (7)..(1362)
  Description of Artificial Sequence Truncated Itk construct
ggatccatgcctgaagaaactgtggtcattgccttatatgactaccaaaccaatgatcctcaggaactcgcactgcggcgcaacgaagagtactgcctgctggacagttctgagattcactggtggagagtccaggacaggaatgggcatgaaggatatgtaccaagcagttatctggtggaaaaatctccaaataatctggaaacctatgagtggtacaataagagtatcagccgagacaaagctgaaaaacttcttttggacacaggcaaagaaggagccttcatggtaagggattccaggactgcaggaacatacaccgtgtctgttttcaccaaggctgttgtaagtgagaacaatccctgtataaagcattatcacatcaaggaaacaaatgacaatcctaagcgatactatgtggctgaaaagtatgtgttcgattccatccctcttctcatcaactatcaccaacataatggaggaggcctggtgactcgactccggtatccagtttgttttgggaggcagaaagccccagttacagcagggctgagatacgggaaatgggtgatcgacccctcagagctcacttttgtgcaagagattggcagtgggcaatttgggttggtgcatctgggctactggctcaacaaggacaaggtggctatcaaaaccattcgggaaggggctatgtcagaagaggacttcatagaggaggctgaagtaatgatgaaactctctcatcccaaactggtgcagctgtatggggtgtgcctggagcaggcccccatctgcctggtgtttgagttcatggagcacggctgcctgtcagattatctacgcacccagcggggactttttgctgcagagaccctgctgggcatgtgtctggatgtgtgtgagggcatggcctacctggaagaggcatgtgtcatccacagagacttggctgccagaaattgtttggtgggagaaaaccaagtcatcaaggtgtctgactttgggatgacaaggttcgttctggatgatcagtacaccagttccacaggcaccaaattcccggtgaagtgggcatccccagaggttttctctttcagtcgctatagcagcaagtccgatgtgtggtcatttggtgtgctgatgtgggaagttttcagtgaaggcaaaatcccgtatgaaaaccgaagcaactcagaggtggtggaagacatcagtaccggatttcggttgtacaagccccggctggcctccacacacgtctaccagattatgaatcactgctggaaagagagaccagaagatcggccagccttctccagactgctgcgtcaactggctgaaattgcagaatcaggactttagggatcc

SEQ ID NO: 7 - protein, 451 aa, Artificial Sequence
  Description of Artificial Sequence Truncated Itk construct
MPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 8 - protein, 620 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 9 - protein, 395 aa, Homo sapiens
MAAVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSW

SEQ ID NO: 10 - protein, 395 aa, Mus musculus
MAAVILESIFLKRSQQKKKTSPLNFKKRLFLLTVHKLSYYEYDFERGRRGSKKGSIDVEKITCVETVIPEKNPPPERQIPRRGEESSEMEQISIIERFPYPFQVVYDEGPLYVFSPTEELRKRWIHQLKNVIRYNSDLVQKYHPCFWIDGQYLCCSQTAKNAMGCQILENRNGSLKPGSSHRKTKKPLPPTPEEDQILKKPLPPEPTAAPISTTELKKVVALYDYMPMNANDLQLRKGEEYFILEESNLPWWRARDKNGQEGYIPSNYITEAEDSIEMYEWYSKHMTRSQAEQLLKQEGKEGGFIVRDSSKAGKYTVSVFAKSTGEPQGVIRHYVVCSTPQSQYYLAEKHLFSTIPELINYHQHNSAGLISRLKYPVSKQNKNAPSTAGLGYGSW

SEQ ID NO: 11 - protein, 356 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKW

SEQ ID NO: 12 - protein, 361 aa, Mus musculus
MNNFILLEEQLIKKSQQKRRTSPSNFKVRFFVLTKASLAYFEDRHGKKRTLKGSIELSRIKCVEIVKSDISIPCHYKYPFQTLVYLQVVHDNYLLYVFAPDCESRQRWVLTLKEETRNNNSLVSKYHPNFWMDGRWRCCSQLEKPAVGCAPYDPSKNASKKPLPPTPEDNRRSFQEPEETLVIALYDYQTNDPQELALRCDEEYYLLDSSEIHWWRVQDKNGHEGYAPSSYLVEKSPNNLETYEWYNKSISRDKAEKLLLDTGKEGAFMVRDSRTPGTYTVSVFTKAIISENPCIKHYHIKETNDSPKRYYVAEKYVFDSIPLLIQYHQYNGGGLVTRLRYPVCSWRQKAPVTAGLRYGKW

SEQ ID NO: 13 - protein, 363 aa, Homo sapiens
MNFNTILEEILIKRSQQKKKTSPLNYKERLFVLTKSMLTYYEGRAEKKYRKGFIDVSKIKCVEIVKNDDGVIPCQNKYPFQVVHDANTLYIFAPSPQSRDLWVKKLKEEIKNNNNIMIKYHPKFWTDGSYQCCRQTEKLAPGCEKYNLFESSIRKALPPAPETKKRRPPPPIPLEEEDNSEEIVVAMYDFQAAEGHDLRLERGQEYLILEKNDVHWWRARDKYGNEGYIPSNYVTGKKSNNLDQYEWYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPVSVKGKNAPTTAGFSYEKW

SEQ ID NO: 14 - protein, 362 aa, Mus musculus
MNFNTILEEILIKRSQQKKKTSLLNYKERLCVLPKSVLSYYEGRAEKKYRKGVIDISKIKCVEIVKNDDGVIPCQNKFPFQVVHDANTLYIFAPSPQSRDRWVKKLKEEIKNNNNIMIKYHPKFWADGSYQCCRQTEKLAPGCEKYNLFESSIRKTLPPAPEIKKRRPPPPIPPEEENTEEIVVAMYDFQATEAHDLRLERGQEYIILEKNDLHWWRARDKYGSEGYIPSNYVTGKKSNNLDQYEWYCRNTNRSKAEQLLRTEDKEGGFMVRDSSQPGLYTVSLYTKFGGEGSSGFRHYHIKETATSPKKYYLAEKHAFGSIPEIIEYHKHNAAGLVTRLRYPVSTKGKNAPTTAGFSYDKW

SEQ ID NO: 15 - protein, 410 aa, Homo sapiens
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIW

SEQ ID NO: 16 - protein, 386 aa, Mus musculus
MESKSILEELLLKKSQQKKKMSPNNYKERLFVLTKTSLSYYEYDKMKRGSRKGSIEIKKIRCVEKVNLEEQTPVERQYPFQIVYKDGLLYVYASNEESRCQWLKALQKEIRGNPHLLIKYHSGFFVDGKFLCCQQSCKAAPGCTLWEAYADLHIAISDEKHRAPTFPERLLKIPRAVPVLKMDASSSGAILPQYDSYSKKSCGSQPTSNIRYIPREDCPDWWQVRKLKSEEDIACSNQLERNIASHSTSKMSWGFPESSSSEEEENLHAYDWFAGNISRSQSEQLLRQKGKEGAFMVRNSSQMGMYTVSLFSKAVNDKKGTVKHYHVHTNAENKLYLAENYCFDSIPKLIHYHQHNSAGMITRLRHPVSTKANKVPVSVALGSGIW

SEQ ID NO: 17 - protein, 264 aa, Homo sapiens
MILSSYNTIQSVFCCCCCCSVQKRQMRTQISLSTDEELPEKYTQHRRPWLSQLSNKKQSNTGRVQPSKRKPLPPLPPSEVAEEKIQVKALYDFLPREPCNLALRRAEEYLILEKYNPHWWKARDRLGNEGLIPSNYVTENKITNLEIYEWYHRNITRNQAEHLLRQESKEGAFIVRDSRHLGSYTISVFMGARRSTEAAIKHYQIKKNDSGQWYVAERHAFQSIPELIWYHQHNAAGLMTRLRYPVGLMGSCLPATAGFSYEKW

SEQ ID NO: 18 - protein, 264 aa, Mus musculus
MILSSYSSFQSVLCCCCCRCSVQKRQVRTQISLSREEELSEKHSQRQRPWFAKLMGKTQSNRGGVQPSKRKPLPPLPQEPPDERIQVKALYDFLPREPGNLALKRAEEYLILERCDPHWWKARDRFGNEGLIPSNYVTENRLANLEIYEWYHKNITRNQTERLLRQEAKEGAFIVRDSRHLGSYTISVFTRARRHTQSSIKHYQIKKNDSGQWYITERHLFPSVPELIQYHQYNAAGLISRLRYPIGLLGSCLPATSGFSYEKW

SEQ ID NO: 19 - DNA, 36 bp, Artificial Sequence
  Description of Artificial Sequence 3 prime oligo
gatcggatccgaagcttattggcgagctcaggattc

SEQ ID NO: 20 - DNA, 43 bp, Artificial Sequence
  Description of Artificial Sequence 5 prime oligo
gatcgcggccgcaccatggcagcagcaccagtctccacaagtg

SEQ ID NO: 21 - protein, 454 aa, Artificial Sequence
  Description of Artificial Sequence Translated polypeptide sequence of the truncated Btk construct
MAAAPVSTSELKKVVALYDYMPMNANDLQLRKGDEYFILEESNLPWWRARDKNGQEGYIPSNYVTEAEDSIEMYEWYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPVSQQNKNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 22 - DNA, 1365 bp, Artificial Sequence
  Description of Artificial Sequence Polynucleotide sequence of the truncated Btk construct
atggcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcggaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgctggcatgagaaagcagatgagcgtcccactttcaaaattcttctgagcaatattctagatgtcatggatgaagaatcctga

SEQ ID NO: 23 - protein, 10 aa, Artificial Sequence
  Description of Artificial Sequence Biotinylated peptide
AAAEEIYGEI